(19) United States
(12) Patent Application Publication
(10) Pub. No.: US 2024/0325684 A1
(43) Pub. Date: Oct. 3, 2024

(54) URINARY CATHETER AND METHODS OF MANUFACTURE

(71) Applicant: The Flume Catheter Company Limited, Farnham (GB)

(72) Inventors: John Havard, Farnham (GB); Roger Anthony Holmes, Farnham (GB)

(21) Appl. No.: 18/513,525

(22) PCT Filed: May 19, 2022
(86) PCT No.: PCT/GB2022/051265

§ 371 Date: Nov. 18, 2023

(30) Foreign Application Priority Data
May 19, 2021 (GB) 2107187.3

(51) Int. Cl. A61M 25/00; A61M 25/10

(52) U.S. Cl. CPC A61M 25/0017; A61M 25/0009; A61M 25/1025

(57) ABSTRACT

A catheter comprising: a shaft (1) having a proximal region and a distal region, the distal region terminating in a tip (4) 12; a balloon located at a distal end of the shaft; an inflation opening (5) located in the distal region of the shaft, the inflation opening communicating with an inflation lumen of the shaft; a connection tube (91) located in the distal region of the shaft, the connection tube communicating with the inflation opening and the interior of the balloon; wherein the connection tube protrudes from the inflation opening.

### FIELD OF THE INVENTION

This invention relates to urinary catheters.

### BACKGROUND

Urinary catheters are used to assist or control the flow of urine from the bladder of a patient. When a patient needs to use a catheter for an extended period of time, they may use an indwelling urinary catheter. An indwelling urinary catheter has a tube which is introduced through the patient's urethra or directly via an abdominal incision (supra-pubic catheter). Once the distal tip of the catheter is in the bladder it is retained in position by means such as a balloon inflated within the bladder. A lumen extending through the catheter can then drain urine from the bladder.

A common design of indwelling urinary catheter is the Foley catheter. In the Foley catheter, the balloon is toroidal in shape and is located proximally of the catheter tip. A drainage opening which communicates with the lumen is located between the catheter tip and the balloon. Catheters of this design suffer from a number of problems. The tip of the catheter is exposed and can irritate the bladder wall. Material of the bladder wall can become drawn into the drainage opening, causing discomfort and mucosal damage. The drainage opening is spaced from the base of the bladder by the balloon, which prevents the bladder draining completely leading to a residual pool of urine that can become infected.

WO 2018/134591 discloses one approach for addressing at least some of these problems. It provides a urinary catheter having an inflatable balloon in the form of an elongate tube extending over the tip and providing the drainage opening on a side of the shaft.

However, this approach may be open to further improvement. To remove the catheter from the patient, the balloon must be uninflated by extracting fluid from the balloon. If the fluid extraction system were to fail, for example if the inflation lumen were to become blocked, it might leave the balloon inflated. In the worst case, this might require access to the bladder through an abdominal incision to burst the balloon.

To attach the balloon to the inflation opening of the shaft, an area of the balloon may be directly attached to the inflation opening, as shown in WO 2018/134591. However, this may result in a complex configuration in order to route the inflation path inside the double skin of the tubular balloon and may create a point stress around the bonded area, which may, depending on the materials used and the manufacturing process, reduce the strength of the balloon. This may reduce the reliability of the catheter.

Catheters may be manufactured by creating a tube into which the drainage and/or inflation openings are formed by piercing the tube. Piercing the openings can result in a rough surface finish on the catheter, particularly when a polyurethane catheter material is used. The rough surface finish can result in discomfort to the patient upon insertion and/or may increase the likelihood of bacteria adhering to the catheter.

There is a need for an improved design and improved manufacturing process of urinary catheters.

### SUMMARY OF THE INVENTION

According to one aspect of the present invention there is provided a catheter comprising: a shaft having a proximal region and a distal region, the distal region terminating in a tip, the shaft having a lumen extending to the tip, the lumen being defined by flanking walls of the shaft and closed by a terminal wall extending across the lumen at the tip; and a balloon defined by sheet material located distally of the terminal wall; wherein the distal part of the lumen has a non-uniform radius perpendicular to the longitudinal axis of the shaft.

The material thickness of the terminal wall may be less than 250% of the material thickness of the flanking walls in the distal region and/or the material thickness terminal wall is less than 50% of the greatest diameter of the shaft in the distal region.

In some embodiments, the catheter may be configured wherein the material thickness of the terminal wall is less than 200% of the material thickness of the flanking walls in the distal region.

In some embodiments, the catheter may be configured wherein the material thickness of the terminal wall is less than 40% of the greatest diameter of the shaft in the distal region.

In some embodiments, the catheter may be configured wherein the material of the shaft has a young's modulus greater than 5 MPa.

In some embodiments, the catheter may be configured wherein the lumen of the shaft is a drainage lumen of the shaft.

In some embodiments, the catheter may be configured to further comprise a drainage opening located in the distal region of the shaft, the drainage opening communicating with the drainage lumen of the shaft.

In some embodiments, the catheter may be configured wherein, in the plane normal to the longitudinal axis of the shaft, the cross-sectional area of the terminal wall is equal to or less than the cross-sectional area of the shaft in the distal region.

In some embodiments, the catheter may be configured wherein, in the plane normal to the longitudinal axis of the shaft, the cross-sectional area of the terminal wall is between 25% and 75% of the cross-sectional area of the shaft in the distal region.

In some embodiments, the catheter may be configured wherein the longitudinal axis of the shaft intersects the terminal wall.

In some embodiments, the catheter may be configured wherein the longitudinal axis of the shaft intersects a centroid of the terminal wall.

In some embodiments, the catheter may be configured wherein the balloon is configured such that when inflated an interior wall of the balloon bears against the tip of the catheter.

In some embodiments, the catheter may be configured wherein the balloon is configured such that when inflated an exterior wall of the balloon is spaced from the tip of the catheter.

According to another aspect of the present invention there is provided a method for manufacturing a catheter comprising: providing a shaft having a proximal region and a distal region, the distal region terminating in a tip, the shaft having a lumen extending to the tip, the lumen being defined by flanking walls of the shaft; introducing a mandrel tool to the lumen at the tip; heating the distal region of the shaft until the distal region is in a plastic state; pressing the distal region of the shaft against a female former; forming a terminal wall between the mandrel and the female former, the terminal wall extending across the lumen at the tip to close the lumen; wherein the distal part of the lumen has a non-uniform radius perpendicular to the longitudinal axis of the shaft.

The method may further comprise thinning the terminal wall around the mandrel tool. The material thickness of the terminal wall may be less than 250% of the material thickness of the flanking walls in the distal region and/or the material thickness terminal wall is less than 50% of the greatest diameter of the shaft in the distal region.

In some embodiments, the method may be configured wherein the material thickness of the terminal wall is less than 200% of the material thickness of the flanking walls in the distal region.

In some embodiments, the method may be configured wherein the material thickness of the terminal wall is less than 40% of the greatest diameter of the shaft in the distal region.

In some embodiments, the method may be configured wherein the material of the shaft has a Young's modulus greater than 5 MPa.

In some embodiments, the method may be configured wherein, in the plane normal to the longitudinal axis of the shaft, the cross-sectional area of the terminal wall is equal to or less than the cross-sectional area of the shaft in the distal region.

In some embodiments, the method may be configured wherein, in the plane normal to the longitudinal axis of the shaft, the cross-sectional area of the terminal wall is between 25% and 75% of the cross-sectional area of the shaft in the distal region.

In some embodiments, the method may be configured wherein the longitudinal axis of the shaft intersects the terminal wall.

In some embodiments, the method may be configured wherein the longitudinal axis of the shaft intersects a centroid of the terminal wall.

The terminal wall may define a surface of an internal cavity of the tip, the internal cavity having a non-uniform radius perpendicular to the longitudinal axis of the catheter shaft.

According to another aspect of the present invention there is provided a catheter comprising: a shaft having a proximal region and a distal region, the distal region terminating in a tip; a balloon located at a distal end of the shaft; an inflation opening located in the distal region of the shaft, the inflation opening communicating with an inflation lumen of the shaft; a connection tube located in the distal region of the shaft, the connection tube communicating with the inflation opening and the interior of the balloon; wherein the connection tube protrudes from the inflation opening.

The connection tube may protrude externally from the inflation opening. The connection tube may be a separate component from the shaft.

In some embodiments, the catheter may be configured wherein the connection tube protrudes at least 2 mm from the inflation opening.

In some embodiments, the catheter may be configured wherein the connection tube protrudes into the interior of the balloon.

In some embodiments, the catheter may be configured wherein the interior of the balloon is secured to the connection tube.

In some embodiments, the catheter may be configured wherein the connection tube protrudes into the interior of the balloon by at least 2 mm.

In some embodiments, the catheter may be configured wherein at least a part of the connection tube is located inside the inflation lumen.

In some embodiments, the catheter may be configured wherein the connection tube extends into the inflation lumen by at least 2 mm.

In some embodiments, the catheter may be configured wherein the shaft comprises a first shoulder in the distal region of the shaft, the first shoulder being recessed relative to the external diameter of the shaft.

In some embodiments, the catheter may be configured wherein the inflation opening is located on the first shoulder of the shaft.

In some embodiments, the catheter may be configured wherein the balloon comprises a first region secured to the connection tube, a second region secured to the shaft and an elastic-walled and/or flexible-walled conduit extending between the first region and the second region.

In some embodiments, the catheter may be configured wherein the shaft comprises a second shoulder on an opposing side of the shaft to the first shoulder, the second shoulder being recessed relative to the external diameter of the shaft, the second region of the balloon being secured to the second shoulder.

In some embodiments, the catheter may be configured wherein the conduit extends over the tip of the shaft.

In some embodiments, the catheter may be configured to further comprise a drainage opening located at the distal end of the shaft, the drainage opening communicating with a drainage lumen of the shaft.

In some embodiments, the catheter may be configured wherein the drainage opening of the shaft is located on a side of the shaft.

In some embodiments, the catheter may be configured wherein at least part of the first region and at least part of the second region are located proximally of the drainage opening.

In some embodiments, the catheter may be configured wherein the first region is at one end of the tube and the second region is at the other end of the tube.

The catheter may be a urinary catheter. In some embodiments, the catheter may be an indwelling urinary catheter configured to be retained in the bladder of a patient.

According to another aspect of the present invention there is provided a method for manufacturing a catheter comprising: providing a shaft having a proximal region and a distal region, the distal region terminating in a tip, a drainage opening located in the distal region of the shaft, the drainage opening communicating with a drainage lumen of the shaft and an inflation opening located in the distal region of the shaft, the inflation opening communicating with an inflation lumen of the shaft; providing a connection tube; securing the connection tube inside the inflation opening; providing an elastic-walled conduit having an access opening to the interior thereof; securing the access opening of the conduit around the connection tube.

In some embodiments, the method may be configured wherein the connection tube protrudes at least 2 mm from the inflation opening.

In some embodiments, the method may be configured wherein the connection tube protrudes into the interior of the conduit.

In some embodiments, the method may be configured wherein the interior of the conduit is secured to the connection tube.

In some embodiments, the method may be configured wherein the connection tube protrudes into the interior of the conduit by at least 2 mm.

In some embodiments, the method may be configured wherein at least a part of the connection tube is located inside the inflation lumen.

In some embodiments, the method may be configured wherein the connection tube extends into the inflation lumen by at least 2 mm.

According to a further aspect of the present invention there is provided a method for manufacturing a catheter comprising: providing a shaft having a proximal region and a distal region, the distal region terminating in a tip, the shaft having a drainage lumen extending to the tip, the drainage lumen being defined by walls of the shaft; introducing a first tool to a wall of the shaft in the distal region; piercing the wall of the shaft in the distal region with the first tool to form a drainage opening communicating with the drainage lumen of the shaft; introducing a second tool into the drainage opening; subjecting the distal region of the shaft to a temperature increase so as to smooth the drainage opening around the second tool.

In some embodiments, the method may be configured wherein introducing the second tool into the drainage opening increases the size of the drainage opening.

In some embodiments, the method may be configured to further comprise, before the temperature increase, introducing a third tool around the tip of the shaft, wherein subjecting the distal region of the shaft to a temperature increase smooths the tip of the shaft.

In some embodiments, the method may be configured wherein the temperature increase raises the temperature of the distal region of the shaft above the softening temperature of the shaft material.

In some embodiments, the method may be configured to further comprise, after the temperature increase, subjecting the distal region of the shaft to a temperature decrease.

In some embodiments, the method may be configured wherein the temperature decrease lowers the temperature of the distal region below the softening temperature of the shaft material.

In some embodiments, the method may be configured wherein the catheter material is held at the increased temperature for a predetermined period of time.

In some embodiments, the method may be configured wherein the predetermined period of time is sufficient to cause the outer surface of the material to smooth.

In some embodiments, the method may be configured wherein the catheter material is a polymer.

In some embodiments, the method may be configured wherein the catheter material is a thermoplastic polymer.

In some embodiments, the method may be configured wherein the shaft has an inflation lumen extending to the tip.

In some embodiments, the method may be configured to further comprise, before the temperature increase, introducing a fourth tool to a wall of the shaft and piercing the wall of the shaft in the distal region with the fourth tool to form an inflation opening communicating with the inflation lumen of the shaft.

In some embodiments, the method may be configured to further comprise, after forming the inflation opening and before the temperature increase, introducing a fifth tool into the inflation opening, wherein subjecting the distal region of the shaft to a temperature increase smooths the inflation opening around the fifth tool.

In some embodiments, the method may be configured wherein the drainage opening is located on the side of the shaft.

In some embodiments, the method may be configured to further comprise securing a balloon to a distal end of the shaft.

In some embodiments, the method may be configured wherein the catheter is an indwelling urinary catheter configured to be retained in the bladder of a patient.

### BRIEF DESCRIPTION OF THE DRAWINGS

The present invention will now be described by way of example with reference to the accompanying drawings. In the drawings:

FIG. 1 is an isometric view of an example of a urinary catheter without a balloon in place.

FIG. 2 is a cross-section of the shaft of the catheter of FIG. 1 on line A-A.

FIG. 3 is a cross-section of the distal part of catheter of FIG. 1 on line B-B, with a partially inflated balloon in place.

FIG. 4 is a cross-section of the distal part of the catheter of FIG. 1 on the line C-C of FIG. 3, with a partially inflated balloon in place.

FIG. 5 is an isometric view of the distal part of the catheter of FIG. 1, with a partially inflated balloon in place.

FIG. 6 is a cross-section of the distal part of catheter of FIG. 1 on line B-B, with a fully inflated balloon in place.

FIG. 7 is a cross-sectional view of the distal part of the catheter of an alternative exemplary embodiment, with a thinned terminal wall and a partially inflated balloon in place.

FIG. 8 shows steps for manufacturing the alternative embodiment of the catheter of FIG. 7.

FIG. 9 shows an example of a mandrel for forming a catheter tip.

FIG. 10(a) shows a vertical cross-sectional view of an embodiment of a catheter formed using the mandrel of FIG. 9.

FIG. 10(b) shows a sectional view along D-D in FIG. 10(a).

FIG. 10(c) shows a sectional view along E-E in FIG. 10(a).

FIG. 11 is a cross-sectional view of the distal part of the catheter of an alternative exemplary embodiment, with a connection tube and a partially inflated balloon in place.

FIG. 12 shows steps for manufacturing the alternative embodiment of the catheter of FIG. 11.

FIG. 13 shows steps for manufacturing a catheter of any of the embodiments.

### DETAILED DESCRIPTION

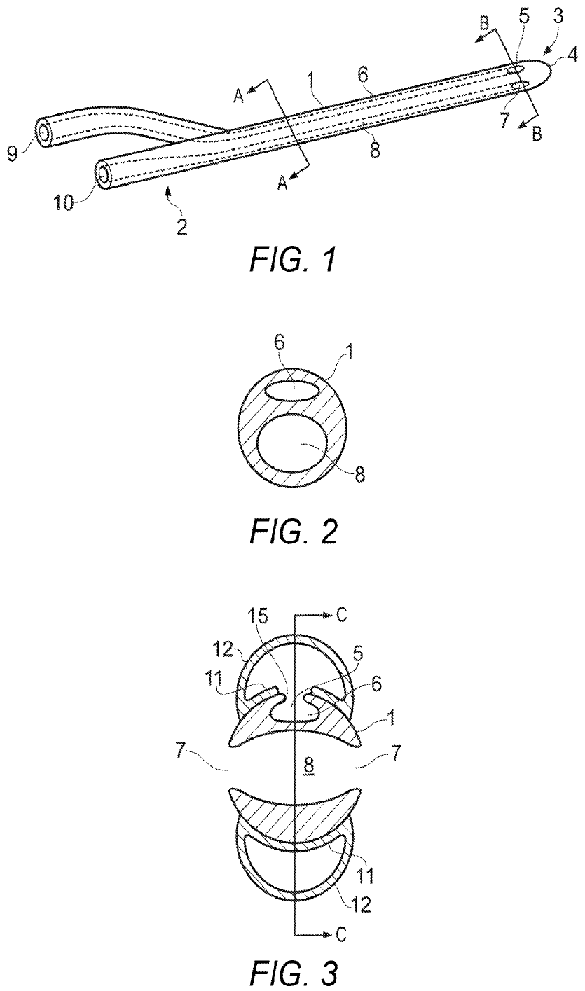

FIG. 1 shows an example of a urinary catheter having a shaft 1. The catheter shaft has a proximal region 2. The proximal region 2 is intended to sit outside the body when the catheter is in use. The catheter has a distal region 3. The distal region 3 is intended to sit in the bladder 17 of a user when the catheter is in use. The distal region 3 of the catheter terminates in a tip 4. Two openings 5, 7 are defined in the distal region 3 of the catheter. An inflation opening 5 is intended for inflating a balloon which can be attached to the catheter. The inflation opening 5 communicates with an inflation lumen 6 which runs along the shaft. A drainage opening 7 is intended for draining urine from the bladder 17 of a user. The drainage opening 7 communicates with a drainage lumen 8 which runs along the shaft. There may be multiple drainage openings 7 in the distal end of the catheter. Preferably each drainage opening 7 communicates with the drainage lumen 8.

FIG. 2 shows a cross-section of the shaft on line A-A of FIG. 1, illustrating the lumens 6, 8. In the proximal region 2 of the shaft 1, the inflation opening 5 communicates with an inflation port 9 and the drainage opening 7 communicates with a drainage port 10. Fluid can be introduced through the inflation port 9 to then pass through the inflation opening 5. Urine received through drainage opening 7 can be collected through drainage port 10. A collecting vessel can be attached to the drainage port 10.

In the examples shown in the figures, the inflation opening 5 and the drainage openings 7 overlap in the longitudinal axis of the catheter. There could be multiple inflation openings 5. The or each inflation opening 5 could be distal of the drainage opening 7, or of a subset of the drainage openings 7 or of all the drainage openings 7. The or each inflation opening 5 could be proximal of the drainage opening 7, or of a subset of the drainage openings 7 or of all the drainage openings 7. Configuring the catheter shaft 1 so that the inflation opening(s) 5 do/does not overlap the drainage opening(s) in a longitudinal direction may help to improve the strength of the shaft.

The entirety of the distal region 3 may taper to the tip 4, or the distal part of the distal region 3 may taper to the tip 4; or the distal region 3 may be of constant diameter about the longitudinal axis of the catheter, in which case the tip 4 may be generally hemispherical.

The balloon may be formed from a continuous tube of elastic material. Preferably, the tube is extruded or drawn into shape. Alternatively, the tube may be formed of a single sheet of material which is folded so its lateral edges meet, the lateral edges then being joined together.

FIG. 3 is a cross-section of the distal region 3 of the shaft on line B-B of FIG. 1, with a partially inflated balloon (not shown in FIG. 1) installed on the shaft.

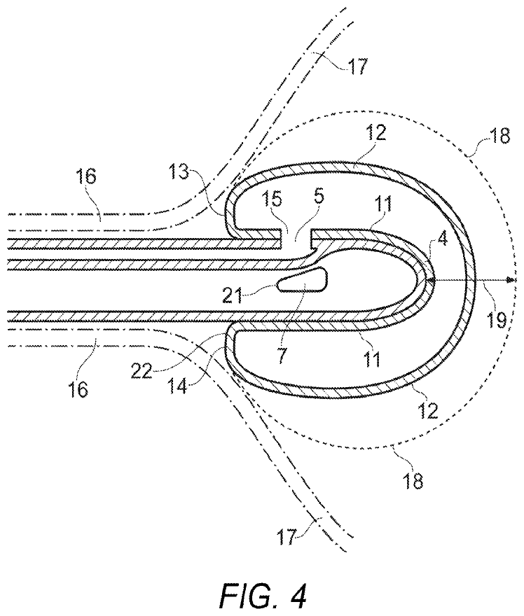

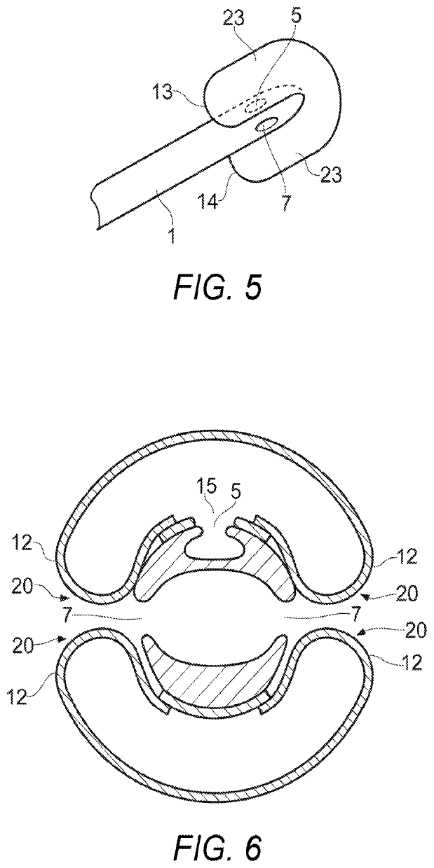

FIG. 4 is a cross-section on line C-C of FIG. 3, and FIG. 5 is an isometric view showing the partially inflated balloon. The catheter of FIG. 2 has two drainage openings 7. In this example, the balloon is generally in the form of a tube having an internal wall 11 and an external wall 12. The tube is generally elongate, extending between ends 13, 14. The balloon is made of an elastic sheet material. The tube constitutes a conduit part or all of whose walls are elastic and/or flexible. The balloon is sealed except for an aperture 15 near one of its ends (end 13), by which the interior of the balloon communicates with the inflation opening 5. The balloon is sealed to the shaft 1 of the catheter around the inflation opening 5. As a result, the balloon can be inflated by introducing fluid such as water or air into the balloon through the aperture 15. The tube-like form of the balloon extends over the tip 4 of the catheter. The balloon is bent around the tip 4. The end 14 of the balloon remote from the aperture 15 is also attached to the distal end 3 of the catheter shaft. This holds the balloon bent over the tip 4.

FIG. 4 shows the balloon in its partially inflated state. FIG. 4 shows in chain-dotted lines the urethra 16 and bladder wall 17 of a person into whom the catheter has been inserted; and dotted line 18 indicates the exterior form of the balloon in its fully inflated state. It should be noted that in its fully inflated state the balloon might be capable of further inflation (i.e. over-inflation). The fully inflated state is the state in which it would normally be left indwelling in a patient's bladder. In its fully inflated state, the size of the balloon, whose outer wall extends radially outward from the shaft of the catheter, resists withdrawal of the catheter through the urethra 16. This retains the distal end 3 of the catheter in the bladder. The balloon can also form a seal at the base of the bladder to resist leakage of urine past the catheter.

Before the catheter is used, a reservoir containing a predetermined volume of fluid can be engaged with the inflation port. The reservoir could be a syringe or a bag. Once the tip of the catheter is in place in the bladder 17, the fluid can be squeezed from the reservoir into the balloon. The predetermined volume of fluid can be such as to cause the balloon to be fully inflated when the reservoir is fully evacuated. A valve may be provided exists in the inflation lumen 6 to resist fluid flow in the inflation lumen 6 towards the proximal region 2 of the catheter. This can help the balloon to remain inflated.

As noted above, the balloon is preferably in the form of an elongate tube folded over the tip 4 of the catheter. In this example, the ends 13, 14 of the tube are attached to the catheter shaft on either side of the distal region of the catheter. In this example, the attachment points are proximal of, and on either side of, the drainage opening 7. In other embodiments the attachment points may overlap the drainage opening(s) 7 or be distal to them. When the balloon is inflated, the sheet material stretches.

The balloon may be attached to the shaft by adhesive, by welding (e.g. thermal welding) or by a mechanical fixing such as a collar configured to clamp the balloon to the exterior of the catheter shaft.

The balloon has an uninflated state. This may be the balloon's state when the catheter is packaged for supply to a user. The catheter having the balloon applied thereto in its uninflated state may be packaged in a sealed package whose interior is sterile.

The balloon may initially adopt the uninflated state. In its uninflated state the exterior surface of the balloon may conform closely to the exterior surface of the catheter. In the uninflated state, the balloon may be taut against the exterior surface of the catheter. This may assist insertion of the catheter into a user.

In its uninflated state the balloon extends over the distal tip 4 of the catheter. One or more regions of the balloon may be attached to the shaft of the catheter. One region of attachment may surround the inflation opening 5. The balloon may have an aperture 15 in its wall facing the inflation opening 5. The aperture 15 may communicate with the inflation opening 5. In this way the balloon can be sealed around the inflation opening 5 to permit pressure in the balloon to be increased by fluid flow through the inflation opening 5.

There may be one, two or more drainage openings 7. Preferably, there is a drainage opening 7 between each leg of the balloon as it extends along the side of the catheter shaft. There may be one, two or more inflation openings 5. The balloon may be inflated from a single end or from more than one end.

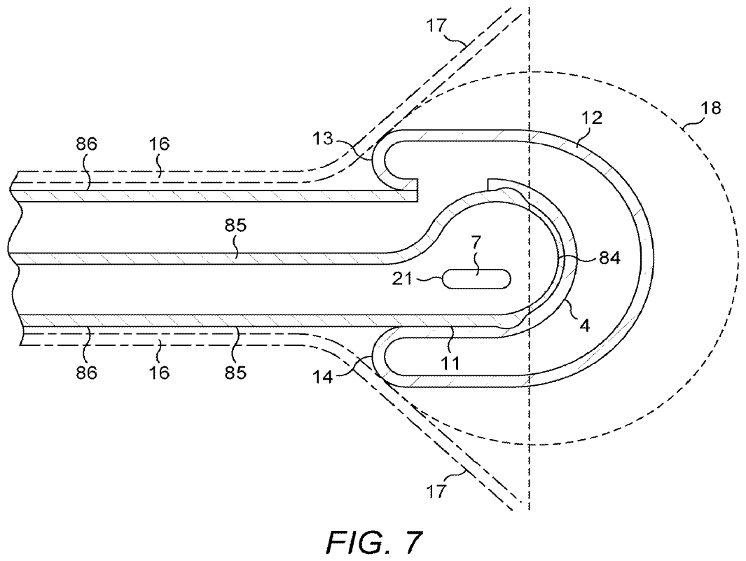

FIG. 7 is a cross-sectional view of the distal region 3 of a catheter of an alternative embodiment. The catheter in this embodiment may comprise any of the features described with respect to the above embodiments.

As mentioned in relation to the previous embodiments described above, the catheter shaft 1 has a proximal region 2 and a distal region 3. The distal region 3 of the shaft 1 terminates in a tip 4. The catheter shaft 1 has at least one lumen 6,8 that extends from the proximal region 2 to the distal region 3. The lumen 6,8 is situated inside the shaft 1 and runs generally parallel to the shaft 1.

A boundary of each of the lumens 6,8 is defined by a wall of the shaft 1. In this embodiment, the walls of the shaft 1 are provided by the shaft itself. It would also be possible for one or more of the lumens 6,8 to be provided by a separate tube that is inserted into the shaft 1.

In particular, the drainage lumen 8 is defined by flanking walls 85 extending along the length of the shaft 1 and a terminal wall 84 at the tip 4 in the distal region 3 of the of the shaft 1.

The flanking walls 85 are generally parallel to the outer walls 86 of the shaft 1 so that the material thickness of the shaft 1 is constant along the length of the shaft 1. This is preferable as a constant shaft thickness may be easier to manufacture. It is also possible for the shaft thickness to vary along the length of the shaft depending on the design requirements.

The terminal wall 84 extends across the lumen 8 at the tip 4 so that the lumen 8 is enclosed at the distal end of the shaft 1. As mentioned above, the lumen 8 also comprises at least one opening 7, so the lumen 8 is not completely enclosed in the distal region 3 but is enclosed at the tip 4 at the distal end of the shaft 1. FIG. 7 shows an inflation lumen 6 and a drainage lumen 8 and an inflation opening 5 and a drainage opening 8 in the distal region 3. In both the inflation and drainage systems the opening 5,7 is located proximal of the terminal wall 84. In this way the lumen 8 is enclosed at the distal end of the shaft 1 but is not fully enclosed in the distal region 3.

FIG. 7 shows the balloon located distally of the terminal wall 84. In this particular example, the balloon is generally in the form of a tube having an internal wall 11 and an external wall 12. The tube is generally elongate, extending between ends 13, 14. The balloon is made of an elastic sheet material. The tube constitutes a conduit part or all of whose walls are elastic and/or flexible. The balloon is sealed except for an aperture 15 near one of its ends (end 13), by which the interior of the balloon communicates with the inflation opening 5. The balloon is sealed to the shaft 1 of the catheter around the inflation opening 5. As a result, the balloon can be inflated by introducing fluid such as water or air into the balloon through the aperture 15. The tube-like form of the balloon extends over the tip 4 of the catheter. The balloon is bent around the tip 4. The end 14 of the balloon remote from the aperture 15 is also attached to the distal end of the catheter shaft 1. This holds the balloon bent over the tip 4.

The shaft 1 may taper in the distal region 3 towards the tip 4, or, as shown in FIG. 7, the distal part of the distal region 3 may taper towards the tip 4. In this embodiment, the greatest diameter of the shaft 1 in the distal region 3 is located at the proximal end of the distal region 3 and the shaft 1 tapers after this point. It is also possible for the distal region 3 to be of constant diameter about the longitudinal axis of the catheter. It is preferable for the shaft 1 to taper in the distal region 3. In particular, it preferable for the taper to increase at the tip 4 to produce a rounded shape. The rounded shape, as shown in FIG. 7, has a number of advantages. Firstly, as mentioned above, the balloon may bend over the tip 4 of the shaft and a rounded shape may provide a continuous surface, without sharp edges, for the balloon to rest against. This way the balloon is less likely to be punctured by accident. Secondly, when the catheter is inserted into the patient, a rounded shape is less likely to catch the urethra 16 and bladder walls 17. This may provide better comfort to the patient.

In the proximal region 2 of the shaft 1, the lumen 6,8 is open through a port 9,10, as shown in FIG. 1. This may allow the lumen 6,8 to communicate with a separate component. This may also provide an aperture for accessing the inside of the lumen 6,8.

When the catheter is in use and has been inserted into the patient, the balloon is filled with a fluid, as explained above, to retain the catheter in the bladder 17 of the patient. To remove the catheter from the patient, the balloon must be uninflated by extracting the fluid from the balloon. In some circumstances the fluid extraction system can fail leaving the balloon inflated. To manually deflate the balloon, a tool may be provided into the lumen 8, through the port 10 in the proximal region 2 of the shaft 1. The tool is then pushed up the lumen 8 along the length of the shaft. The tool is pushed into a wall, for example the terminal wall 84, to perforate the terminal wall 84. As the balloon is located distally of the terminal wall 84, once the tool is pushed through the terminal wall 84 it will be pushed into the balloon and cause the balloon to perforate and burst.

The terminal wall of the tip of a conventional catheter is often considerably thicker than the flanking walls due to the forming process used during manufacturing. During the processing of a conventional polyurethane catheter tip, an open-ended tube is closed by introducing a mandrel into the drainage lumen and heating the tip against a correspondingly shaped female mould. This closes the open end of the tube. As a result of this process, a mass of solidified material is present at the distal end of the tip and the material thickness of the terminal wall at the tip of the catheter is usually much thicker than the thickness of the flanking walls in the distal region.

Due to the perforation requirement mentioned above, it is preferable to provide a thin terminal wall 84.

In this embodiment, the material thickness of the terminal wall 84 is sufficiently thin to allow perforation by a tool. The material thickness of the terminal wall 84 may be less than 250% of the material thickness of the flanking walls 85. Alternatively, the material thickness of the terminal wall 84 may be less than 50% of the material thickness of the greatest diameter of the shaft 1 in the distal region 3. As described above, the diameter of the shaft 1 in the distal region 3 may be constant or may vary along the longitudinal axis of the shaft 1. In this embodiment, the diameter of the shaft 1 tapers in the distal region 3. By reducing the thickness of the terminal wall 84 it means that the terminal wall 84 may require less force to perforate. By requiring less force to perforate, the thinner terminal wall 84 may enable a less sharp, less stiff and/or less thick tool to be used. A less sharp tool may reduce the risk of the tool perforating the incorrect section of the shaft 1 as the tool is pushed up the shaft 1. A less stiff tool may enable the tool to more easily follow the curvature of the urethra tract as the tool is pushed up the shaft 1. A less thick tool may enable a protective sleeve to encase the tool to also reduce the risk of the tool perforating the incorrect section of the shaft 1 as the tool is pushed up the shaft 1.

Alternatively, the material thickness of the terminal wall 84 may be less than 200%, less than 150%, less than 100%, less than 80% or less than 50% of the material thickness of the flanking walls 85. Alternatively, the material thickness of the terminal wall 84 may be less than 40%, less than 35% or less than 30% of the material thickness of the greatest diameter of the shaft 1 in the distal region 3. This way, the force required to perforate the terminal wall may be further reduced.

The material thickness of the terminal wall may be uniform across the terminal wall. Alternatively it may vary. Where the material thickness of the terminal wall varies, the thickness over the entire tip may meet the criteria identified above. The thickness of the flanking walls may be uniform or may vary. Where the material thickness of the flanking walls varies, the material thickness of the terminal wall may satisfy the first criterion identified above in comparison to all parts of the flanking walls. The diameter of the shaft in the distal region may be uniform or may vary. Where the diameter of the shaft in the distal region varies, the material thickness of the terminal wall may satisfy the second criterion identified above in comparison to all diameters of the shaft in the distal region.

It is also preferable that the shaft 1 material comprises a Young's modulus of more than 15 MPa. The material stiffness of the shaft 1 should be sufficient to avoid the shaft 1 bending or kinking when inserted into the patient while also being low enough to allow the terminal wall 84 to be perforated.

The shaft 1 of the catheter may be formed of a material such as polyurethane, a silicone elastomer or latex. A polyurethane catheter shaft 1 can be more rigid than comparable rubber or latex catheter shafts. This may allow the shaft to have a larger urine carrying capacity without sacrificing rigidity for insertion.

Latex often has a Young's modulus of less than 5 MPa. It is therefore preferable that the shaft 1 comprises polyurethane. Conveniently the shaft 1 stiffness is greater for a given size than a shaft of the same dimensions would be if formed from latex having a Young's modulus of less than 5 MPa.

As the balloon is located distally of the terminal wall 84, is it preferable for the terminal wall 84 to be arranged close to (for example, adjacent to) the balloon so that the tool can be pushed directly through the terminal wall 84 and into the balloon to deflate or burst the balloon. In this embodiment the drainage lumen 8 is positioned closer to the longitudinal axis of the shaft 1, extends further in the distal direction and is wider than the inflation lumen 6. Thus, the distal end of the drainage lumen 8 is closer to the balloon and provides wider access for the tool then the inflation lumen 6. Additionally, in this embodiment, the drainage lumen 8 provides a generally straight route up the catheter whereas the inflation lumen 6 comprises a bend at the distal end. The straight route may be preferable as it may provide an easier route for the tool to follow. The larger diameter of the drainage lumen may also accommodate a protective sheath for the advanced tool. Consequently, in the embodiment shown in FIG. 7, the terminal wall 84 is provided at the end of the drainage lumen 8. However, in another embodiment, the terminal wall could alternatively, or additionally, be provided at the end of the inflation lumen 6.

In this embodiment, it is preferable that the drainage opening 7 is located on the side of the shaft 1 in the distal region 3. As mentioned above, the terminal wall 84 provides a region for perforation by a tool to allow the balloon to be burst in the event that it cannot be deflated by removing fluid through the inflation lumen. When the tool is provided along the drainage lumen 8, the flanking walls 85 may generally direct the tool along the longitudinal axis of the shaft 1 to the terminal wall 84. In this way the perforation part of the tool, i.e. the cutting surface, is unlikely to contact the sides of the shaft 1. Thus, as the drainage opening 7 is located on the side of the shaft 1 it is unlikely that the tool will accidently push out of the drainage opening 7.

Once the tool has reached the perforation region of the drainage lumen 8, provided by the terminal wall 84, the tool can be pushed through the terminal wall 84 to perforate the balloon. As mentioned above, the flanking walls 85 may naturally guide the tool along the longitudinal axis of the shaft 1 towards the terminal wall 84. It is therefore preferable that the terminal wall 84 is positioned close to the longitudinal axis of the shaft 1. In particular, it is preferable for the terminal wall 84 to intersect the longitudinal axis of the shaft 1 so that if flanking walls 85 have guided the tool along the longitudinal axis of the shaft 1 then the tool will perforate the terminal wall 84 as desired.

It is also preferable for the tool to contact the terminal wall 84 in the centre of the terminal wall 84, as this is likely to be the easiest part of the terminal wall 84 to perforate. For example, the rigidity of the terminal wall 84 may reduce as it extends from the surround flanking walls 85. Thus, it is preferable for the longitudinal axis of the shaft 1 to intersect with the centroid of the terminal wall 84. In this way, it is more likely that the tool will contact and perforate the terminal wall 84 at the weakest point, and as mentioned above, this is preferable for safety and usability reasons.

If in fact the tool is not guided by the flanking walls 85, as described above, it might be that the tool does not follow the longitudinal axis of the shaft 1. In this case the point at which the tool contacts the wall of the shaft 1 may vary. The terminal wall 84 may therefore be large enough to allow for any alterations in the contact point of the tool on the wall of the shaft 1

The terminal wall 84 may have the same or smaller cross-sectional area, in a plane normal to the longitudinal axis of the shaft 1, the cross-sectional area of the shaft 1 in the distal region 3. The cross-sectional area of the shaft 1 in the distal region 3 is defined as the largest cross-sectional area in the distal region 3. As mentioned above, the shaft 1 may have a generally constant section along the length but taper in the distal region 3. In this embodiment, the shaft tapers in the distal part of the distal region 3 to form a rounded tip 4. Therefore, in this embodiment, the cross-sectional area of the shaft 1 could be measured at any point along the shaft 1 before the shaft 1 begins to taper.

The cross-section of the terminal wall 84 may be between 25% to 75% of the shaft 1 cross-sectional area. As explained above, in this range the terminal wall 84 is large enough for the tool to easily perforate the terminal wall 84. In the embodiment shown in FIG. 7, the terminal wall 84 is within the 25% to 75% range.

As mentioned above, in this embodiment the balloon extends over the tip 4 of the catheter. The balloon may also bear against the tip 4 of the catheter. As the balloon is located distally of the terminal wall 84 and the terminal wall 84 is at the tip 4, once the terminal wall 84 has been perforated, the tool will then make direct contact with the balloon as the balloon is bearing against the tip 4. This reduces the likelihood of the balloon not being positioned on the outside of the perforated wall and the balloon not being perforated by the tool.

It is also possible for the balloon to be separated from the tip 4. This would result in the tool needing to travel further from the terminal wall 84 to the balloon. Provided the balloon is located in line with the terminal wall 84, along the longitudinal axis of the shaft 1, the balloon will still be perforated by the tool when the tool is pushed through the terminal wall 84.

It may also be appreciated that the above embodiment, as shown in FIG. 7, would apply to the catheter when the catheter shaft 1 is not straight. When the catheter is inserted into the patient, it may be required to bend to fit to the profile of the urethra 16 and the bladder 17. It may therefore also be appreciated that the tool for perforating the terminal wall 84 and the balloon may be flexible to follow the profile of the catheter shaft 1 in the patient.

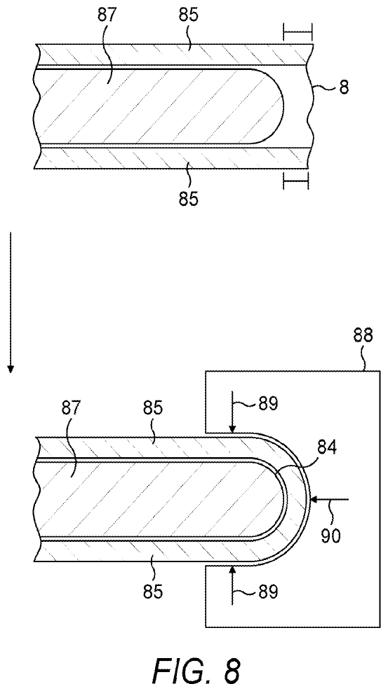

FIG. 8 shows a method of manufacturing the alternative embodiment illustrated in FIG. 7. The manufacturing system comprises a mandrel 87 and a die 88. Die 88 acts as a female former. Both the mandrel 87 and the die 88 may be heated and provide pressure. The method of manufacturing is outlined below.

A catheter shaft 1 is provided with a proximal region 2 and distal region 3. The distal region 3 of the shaft 1 ends in a tip 4. The shaft 1 has at least one lumen 6,8 that extends from the proximal region 2 to the distal region 3. The lumen 8 is defined by flanking walls 85 of the shaft 1.

The shaft 1 may have more than one lumen, for example an inflation lumen 6 and a drainage lumen 8, as described above. The inflation lumen 6 and the drainage lumen 8 may be arranged as described in relation to FIG. 7 above. The method of manufacture described in this embodiment relates to the drainage lumen 8, as it is the drainage lumen that provides the terminal wall 84 for perforation in the embodiment of FIG. 7. It may also be appreciated that the method of manufacture could equally apply to an inflation lumen 6.

A mandrel 87 is introduced along the drainage lumen 8 to the tip 4 of the shaft 1. The mandrel 87 is preferably provided at a distance proximal of the tip 4, as shown in FIG. 8, to aid the forming step below. Preferably this distance is 2 mm. During this step, the die 88 may also be provided around the tip 4 of the shaft 1. The mandrel 87 has preferably the same or a smaller diameter as the drainage lumen 8 so that the drainage lumen 8 is a tight fit around the mandrel 87, but not so tight that the mandrel 87 is not easily inserted or removed. The mandrel 87 may provide a convex rounded, pointed, square or any other suitably shaped convex mould. The die 88 may provide a corresponding concave rounded, pointed, square or any other suitably concave shaped mould.

Once the mandrel 87 and die 88 are in position, the distal region 3 of the shaft 1 is heated. The heat may be provided by the mandrel 87, die 88 or an external heater. The heat is provided until the distal region 3 is in a plastic state. The plastic state may be defined as providing sufficient malleability to enable the forming process below.

The terminal wall 84 is formed around the mandrel 87 to extend across the at the tip 4 to close the drainage lumen 8. The die 88 provides heat and pressure to the flanking wall 85, as shown in FIG. 8, in directions radially towards 89 the longitudinal axis of the shaft 1 and along the longitudinal axis 90 of the shaft 1. This pressure 89,90 pushes the flanking walls 85 together around the mandrel 87 to form the terminal wall 84.

The heating is sufficient to soften the distal region 3 of the shaft 1. In other words, the temperature is raised to a level above the softening temperature of the shaft 1 material. At this level the combination of the pressure from mandrel 87 and die 88 and the softened state of the shaft 1 material is sufficient for the shaft 1 material to flow and follow the surface of the mandrel 87 and die 88.

The terminal wall 84 is thinned around the mandrel 87. The die 88 provides heat and pressure in a direction along the longitudinal axis 90 of the shaft 1. This pressure 90 squeezes the terminal wall 84 which reduces the thickness of the terminal wall 84. This results in the terminal wall 84 being thinner than if it was not squeezed by the mandrel 87 and die 88.

It may be appreciated that the terminal wall 84 may comprise any of the features mentioned above in relation to the embodiment of FIG. 7.

The action of the mandrel 87 against the terminal wall 84 forms an internal cavity at the end of the drainage lumen 8 of the catheter. The internal cavity is distal of the drainage opening(s). The terminal wall 84 closing the lumen 8 at the tip 4 extends across the lumen 8 and defines a surface of the internal cavity. The tip therefore has an internal hollow region at its distal end, at the end of drainage lumen and distal of the drainage opening(s).

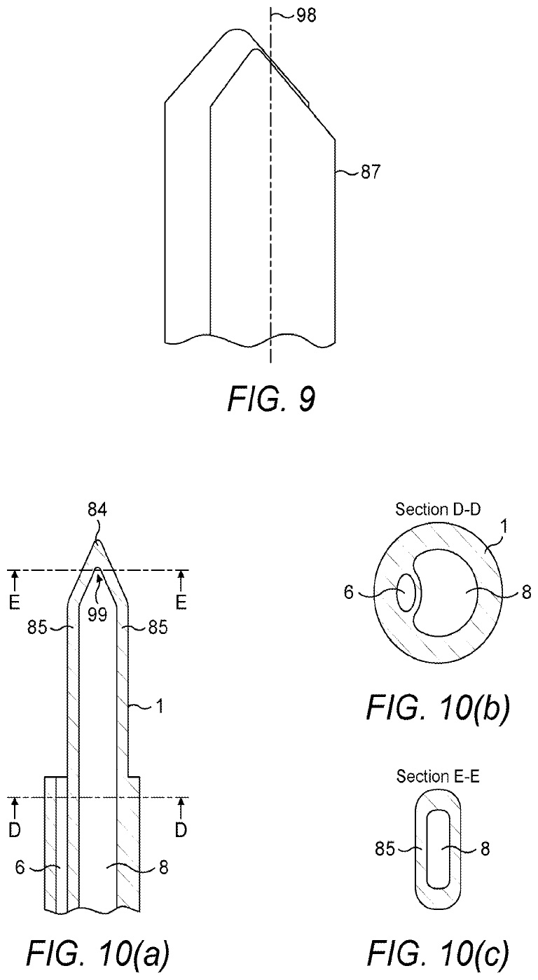

In a preferred embodiment, the mandrel used in the formation of the tip, as described above, is asymmetrically shaped about at least one plane parallel to its longitudinal axis. One example of a mandrel 87 used to form the tip is schematically illustrated in FIG. 9. The longitudinal axis of the mandrel is indicated at 98.

An example of a vertical cross-sectional view of a catheter formed using the mandrel of FIG. 9 and a female die, which in this example has a corresponding shape to the mandrel, is shown in FIG. 10(a). The vertical cross-sectional view of FIG. 10(a) is taken along a plane containing the central longitudinal axis of the catheter shaft 1 and the inflation lumen 6. Horizontal cross sectional views taken along D-D and E-E in FIG. 10(a) are shown in FIGS. 10(b) and 10(c) respectively.

The resulting internal tip cavity 99 of the catheter is asymmetrical about a plane normal to the longitudinal axis of the catheter shaft 1 and/or the inflation lumen 8 of the catheter.

In this example, the internal diameter of the internal tip cavity 99 is smaller along a first axis normal to the longitudinal axis of the catheter shaft than along a second axis normal to the longitudinal axis of the catheter shaft and perpendicular to the first axis. Specifically, the internal diameter of the tip cavity 99 is smaller along a direction parallel to a plane containing the longitudinal axis of the catheter shaft and the inflation lumen 6 than along a direction normal to the plane containing the longitudinal axis of the catheter shaft and the inflation lumen 6.

In the above examples, the material thickness of the terminal wall 84, which defines a surface of the internal cavity 99, may be less than 250% of the material thickness of the flanking walls. Alternatively, the material thickness of the terminal wall may be less than 50% of the material thickness of the greatest diameter of the shaft in the distal region. More preferably the material thickness of the terminal wall 84 may be less than 200%, less than 150%, less than 100% or less than 80% of the material thickness of the flanking walls 85. Alternatively, the material thickness of the terminal wall 84 may be less than 40%, less than 35% or less than 30% of the material thickness of the greatest diameter of the shaft 1 in the distal region 3.

The external diameter of the catheter tip in the distal region may have corresponding features to the internal tip cavity 99. For example, the external diameter of the catheter tip in the distal region may be wider along a first axis normal to the longitudinal axis of the catheter shaft than along a second axis perpendicular to the first axis. Specifically, the external diameter of the catheter tip may be smaller along a direction parallel to a plane containing the longitudinal axis of the catheter shaft and the inflation lumen 6 than along a direction normal to the plane containing the longitudinal axis of the catheter shaft and the inflation lumen 6.

The shape of the catheter tip resulting from this formation process allows the tubular balloon described above to be held in place over the tip of the catheter without the balloon protruding unnecessarily from the profile of the catheter shaft when the balloon is uninflated. Because the external diameter of the tip is wider in one direction than in the direction normal to this, this can provide more frictional resistance to the balloon folded over the tip, as there is a greater surface area of the tip in contact with the balloon along this direction.

Thus, the tip region of the catheter may be formed by the following steps:
A mandrel having a suitably shaped distal end is introduced into a tube. The tube may be open- or closed-ended at its end (“end A”) opposite the end (“end B”) from which the mandrel is introduced.
End A of the tube is positioned against a female former. Heat is applied to end A. Conveniently, this may be done by heating the female former. Heat is applied so that end A becomes at least partially plastic.
End A of the tube is pressed against the female former. If the tube was open-ended at end A, this pressing step may close the end of the tube. As part of the pressing step, pressure is applied through the closed end of the tube against the female former. This causes end A of the tube to adopt a shape such that its exterior fits to the shape of the female former and its interior fits to the shape of the male distal end of the mandrel. In this way, the interior and exterior shapes of the tip can be controlled. The distance to which the mandrel advances into the female former can be controlled so as to set the thickness of the wall of the tube at end A.
Once the tube has cooled and set in shape, the mandrel is removed and the tube is removed from the female former.

As noted above, the cross-section of the mandrel perpendicular to its longitudinal axis in the region where it shapes the distal region of the catheter is not circular. Conveniently, there are two diametrically opposed parts of the cross-section that have a greater radius than two other diametrically opposed parts of the cross-section. For example, the cross-section may be generally elliptical or oval. Or it may have two opposing curved parts joined by two straight walls, the radius being less at the walls than at the curved parts.

The cross-section of the female former where it shapes the distal region of the catheter may follow that of the distal part of the mandrel so that when the distal region of the catheter is sandwiched between the mandrel and the female former it can be given a uniform wall width.

On at least some planes perpendicular to the longitudinal axis of the mandrel, the cross-section of the distal part of the mandrel may conveniently be non-circular.

Conveniently those cross-sections may be of a bi-lobal, oval or elliptical shape, with opposing regions of smaller diameter than adjoining regions of the mandrel. Those adjoining regions may be circumferentially and/or longitudinally adjoining. Regions of minimum radius may occupy greater than 20% or greater than 30% of the circumference of the mandrel on those planes.

The female former may be shaped as an enlarged version of the mandrel. Thus, on at least some planes of the distal region of the resulting catheter perpendicular to the longitudinal axis of the catheter shaft, the cross-section of the distal part of the catheter may conveniently be non-circular. Conveniently those cross-sections may be of a bi-lobal, oval or elliptical shape, with opposing regions of smaller diameter than adjoining regions of the exterior of the catheter. Those adjoining regions may be circumferentially and/or longitudinally adjoining. Regions of minimum radius may occupy greater than 20% or greater than 30% of the circumference of the catheter on those planes.

This process can provide a catheter tip region that (a) is relatively thin, so that it can readily be pierced to deflate a balloon located immediately distally of the tip and/or (b) is of non-uniform radius perpendicular to its longitudinal axis. This latter feature can allow for an uninflated balloon to lie in one or more relatively recessed portions of the tip region. This can avoid the uninflated balloon protruding excessively from the general profile of the catheter shaft. One or more inflation openings can be located at (a) region(s) that have/has a smaller radius about the longitudinal axis of the shaft than other regions. One or more drainage openings can be located at (a) region(s) that have/has a larger radius about the longitudinal axis of the shaft than other regions.

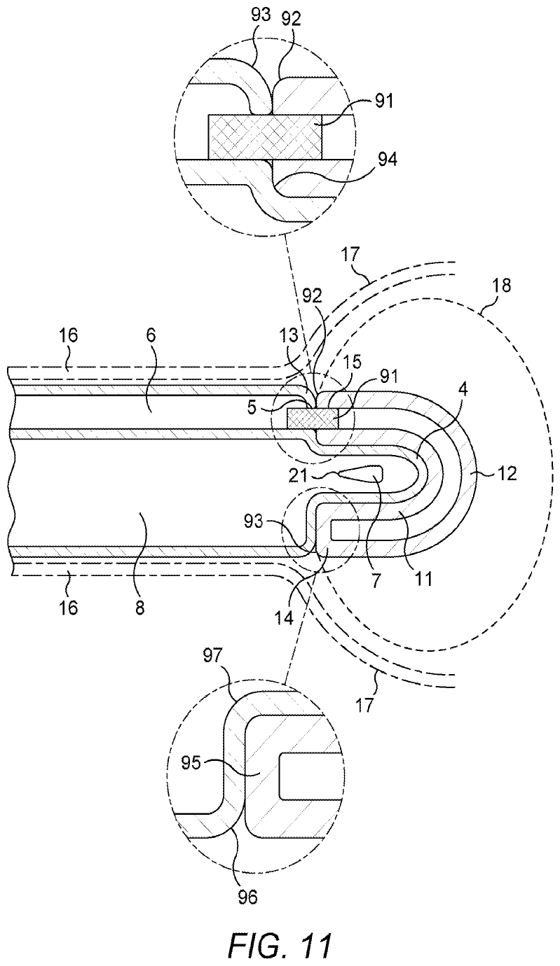

FIG. 11 is a cross-sectional view of the distal region 3 of a catheter of an alternative embodiment. The catheter in this embodiment may comprise any of the features described with reference to the above embodiments.

As mentioned in relation to the previous embodiments above, the catheter shaft 1 has a proximal region 2 and a distal region 3. The distal region 3 of the shaft 1 terminates in a tip 4. The catheter shaft 1 has at least one lumen 6,8 that extends from the proximal region 2 to the distal region 3. The lumen 6,8 is situated inside the shaft 1 and runs generally parallel to the shaft 1.

In this embodiment, the shaft 1 comprises two lumens, an inflation lumen 6 and a drainage lumen 8. As shown in FIG. 11, the inflation lumen 6 is generally positioned to a side of the shaft 1 and the drainage lumen 8 is more central to the longitudinal axis of the shaft. As shown in FIG. 2, the inflation lumen 6 is preferably also smaller than the drainage lumen 8. It is possible for the inflation lumen 6 and drainage lumen 8 to be arranged differently, such as on either side of the longitudinal axis of the shaft 1, and to be of equal size.

The shaft 1 also comprises an inflation opening 5 which is intended for inflating a balloon which can be attached to the catheter. The inflation opening 5 communicates with the inflation lumen 6 which runs along the shaft. The shaft 1 also comprises a drainage opening 7 which is intended for draining urine from the bladder 17 of a user. The drainage opening 7 communicates with the drainage lumen 8 which runs along the shaft. There may be multiple drainage openings 7 in the distal end of the catheter, although only one opening is visible in FIG. 11. Preferably each drainage opening 7 communicates with the drainage lumen 8.

FIG. 11 shows the balloon located at the distal end of the shaft 1. In its preferred form, the balloon is generally in the form of a tube having an internal wall 11 and an external wall 12. The tube is generally elongate, extending between ends or regions 13, 14. The balloon is made of an elastic sheet material. The tube constitutes a conduit part or all of whose walls are elastic and/or flexible.

FIG. 11 shows the balloon in its partially inflated state. FIG. 11 shows in chain-dotted lines the urethra 16 and bladder wall 17 of a person into whom the catheter has been inserted; and dotted line 18 indicates the exterior form of the balloon in its fully inflated state. It should be noted that in its fully inflated state the balloon might be capable of further inflation (i.e. over-inflation). The fully inflated state is the state in which it would normally be left indwelling in a patient's bladder. In its fully inflated state, the size of the balloon, whose outer wall extends radially outward from the shaft of the catheter, resists withdrawal of the catheter through the urethra 16. This retains the distal end of the catheter in the bladder 17. The balloon can also form a seal at the base of the bladder to resist leakage of urine past the catheter.

The balloon is sealed except for an aperture 15 near one of its ends (end 13), by which the interior of the balloon communicates with the inflation opening 5. In this embodiment the aperture 15 is located at the end 13 of the tube in the first region 13. The catheter of this embodiment comprises a connection tube 91 located in the distal region 3 of the shaft 1. The connection tube 91 provides a means for communicating between the inflation opening 5 and the interior of the balloon through the aperture 15. As a result, the balloon can be inflated by introducing fluid such as water or air into the balloon through the aperture 15.

It is advantageous to provide a simplified connection between the inflation opening 5 and the balloon. Traditionally, when a balloon is connected to the inflation opening by bonding the skin of the balloon directly to the inflation opening, this may create a point of stress, which may reduce the strength of the balloon. This may reduce the reliability of the catheter. By simplifying the connection via the use of the connection tube, this may improve the reliability of the catheter. The connection tube conveniently directs the inflation path inside the skin of the balloon. Additionally, the simpler connection may provide an easier manufacturing process.

It is advantageous to provide a more secure connection between the inflation opening 5 and the balloon. When the catheter is inserted into the patient, the urethra 16 and bladder wall 17 can push against and produce a friction force on the tip 4 of the catheter. As the balloon is located distally of the tip 4, this friction force may act on the balloon and result in the balloon being detached from the catheter. If the balloon is detached from the catheter, this can cause discomfort to the patient, and in the worst case result in the balloon being left in the bladder of the patient after removal of the catheter. A more secure connection between the balloon and the inflation opening 5 can reduce the risk of the balloon detaching from the catheter.

The connection tube 91 may be provided as a tube with an inside and outside wall. The cross-section of the connection tube 91 may be circular, ovular, square, rectangular or any other shape. Preferably the connection tube 91 is generally circular as this is the standard form of manufactured tube. The shape may vary depending on the design requirements for the catheter.

The connection tube 91 is in connection with the inflation opening 5 and protrudes from the inflation opening 5. The connection tube 91 also protrudes into the interior of the balloon through the aperture 15. The connection tube 91 protrusion can provide a mating surface for the interior of the balloon to be secured to. The balloon may be secured to the connection tube 91 using adhesive or any other suitable securing means. The mating surface of the connection tube 91 can provide additional area for the interior of the balloon to mate with the connection tube 91. This can provide a larger area for applying adhesive and consequently provide a more secure joint than if the balloon was directly attached to the inflation opening 5. Preferably the connection tube 91 protrudes at least 2 mm from the inflation opening 5 and protrudes at least 2 mm into the interior of the balloon to provide a sufficient mating surface. These dimensions may vary depending on the size of the catheter and the strength of the adhesive.

The connection tube 91, as shown in FIG. 11, may also protrude through the inflation opening 5 and into the inflation lumen 6. This may provide a mating surface for the connection between the connection tube 91 and the inflation lumen 6. The connection tube 91 may be secured to the inflation lumen 6 using adhesive or any other suitable securing means. The mating surface of the connection tube 91 can provide additional area for the inflation lumen 6 to mate with the connection tube 91. This can provide a larger area for applying adhesive and consequently provide a more secure joint than if the balloon was directly attached to the inflation opening 5. Preferably the connection tube 91 protrudes at least 2 mm into the inflation lumen 6 to provide a sufficient mating surface. These dimensions may vary depending on the size of the catheter and the strength of the adhesive.

It is also possible for the connection tube 91 to be an integral part of the catheter shaft 1. In other words, the connection tube 91 is formed as part of the catheter shaft 1 and protrudes from the catheter shaft inflation opening 5. This arrangement may also allow for improved securing of the interior of the balloon to the inflation opening 5 as explained above.

As mentioned above, in this embodiment the aperture 15 is located at the end 13 of the tube in the first region 13. As shown in FIG. 11, a portion of the first region 13, not comprising the aperture 15, can be attached to the distal end 3 of the shaft 1. Securing the first region 13 of the balloon to the shaft 1 can further increase the strength of the connection between the balloon and the inflation opening 5. Similarly, the second region 14 of the balloon can be attached to the distal end 3 of the shaft 1 to further strengthen the connection between the balloon and the shaft 1.

The tube-like conduit of the balloon is formed between the first region 13 and the second region 14 of the balloon. The first region 13 and the second region 14 can be attached on opposing sides of the shaft 1. This way, the tube-like form of the balloon extends over the tip 4 of the catheter. The balloon is bent around the tip 4. By attaching the first region 13 and the second region 14 to the shaft 1 this holds the balloon bent over the tip 4.

As shown in FIG. 11, the shaft 1 may further comprise a first shoulder 92. The first shoulder 92 is located in the distal region 3 of the shaft 1. The first shoulder 92 is recessed relative to the external diameter of the shaft 1. In this example, the first shoulder 92 comprises a first shoulder edge 93 and a first shoulder corner 94. Similarly, the shaft may also comprise a second shoulder 95. The second shoulder 95 is located in the distal region 3 of the shaft 1. The second shoulder 95 is recessed relative to the external diameter of the shaft 1. In this example, the second shoulder 92 comprises a second shoulder edge 96 and a second shoulder corner 97. The first shoulder edge 93 and second shoulder edge 96 may be rounded or square. It is preferable for the edges 93, 96 to be rounded to reduce the likelihood of the edges catching the urethra 16 or bladder 17 wall upon inserting the catheter into the patient. The first shoulder corner 94 and second shoulder corner 97 may also be rounded or square. It is preferable for the corners 94, 97 to be rounded because, as explained below, the balloon may be pushed into the corners 94, 97 and a rounded corner 94, 97 may be less likely to unintentionally perforate the balloon.

The first shoulder 92 and the second shoulder 95 provide a surface for the balloon first region 13 and second region 14 to be respectively attached. As the first shoulder 92 and the second shoulder 95 are recessed relative to the external diameter of the shaft 1, the surface of the shoulders 92, 95 is provided normal to the longitudinal axis of the shaft 1. The first region 13 and the second region 14 of the balloon are provided at the ends 13, 14 of the balloon. In this way, as shown in FIG. 11, the first shoulder 92 and the second shoulder 95 provide a surface for the balloon first region 13 and second region 14 to be respectively attached to the shaft 1. As explained above, the first region 13 and second region 14 are attached to the shaft 1, as well as the first region 13 being attached to the inflation opening 5. By providing a surface on the shoulders 92, 95 this can provide a larger and flatter mating surface for the connection than if the connection was provided on the wall of the shaft 1. This may further increase the strength of the connection.

In FIG. 11, it is shown that the inflation opening 5 is located on the first shoulder 92 of the shaft 1. The result of this is that the connection tube 91 protrudes from the first shoulder 93 and into the interior of the balloon. The combination of attaching the first region 13 to the first shoulder 92 and the connection tube 91 attaching to the interior of the balloon is that the connection between the balloon and the inflation opening 5 may be significantly strengthened.

The first shoulder 93 and the second shoulder 95 also provide a recess in the shaft of the catheter for the first region 13 and the second region 14 of the balloon to be located in. As shown in FIG. 11, the shoulders 93 are recessed from the external diameter of the shaft 1 so that the first region 13 and the second region 14 of the uninflated balloon may also be located in line with (or flush with) or inside the external diameter of the shaft 1. Providing the uninflated balloon inside the external diameter of the shaft 1 reduces the likelihood of protruding features catching on the urethra 16 or the bladder wall 17 when the catheter is inserted into the patient.

In other embodiments, the first shoulder 93 and the second shoulder 95 may in fact provide a continuous ledge around the entire circumference of the shaft 1. In this way, the shaft 1 would have a single continuous shoulder.

As shown in FIG. 11, the drainage opening 7 is located on a side of the shaft 1. The balloon extends from the first and second regions 13, 14 and over the tip 4. In this example the first and second regions 13, 14 are proximal, and on either side, of the drainage opening 7. In other embodiments the attachment points may overlap the drainage opening(s) or be distal to them. The result of this is that the drainage opening 7 is recessed relative to the inflated balloon, as shown in FIG. 6. This reduces the likelihood of the drainage process drawing the patient's bladder wall 17 into the drainage opening 7, which may cause significant discomfort and possible injury.

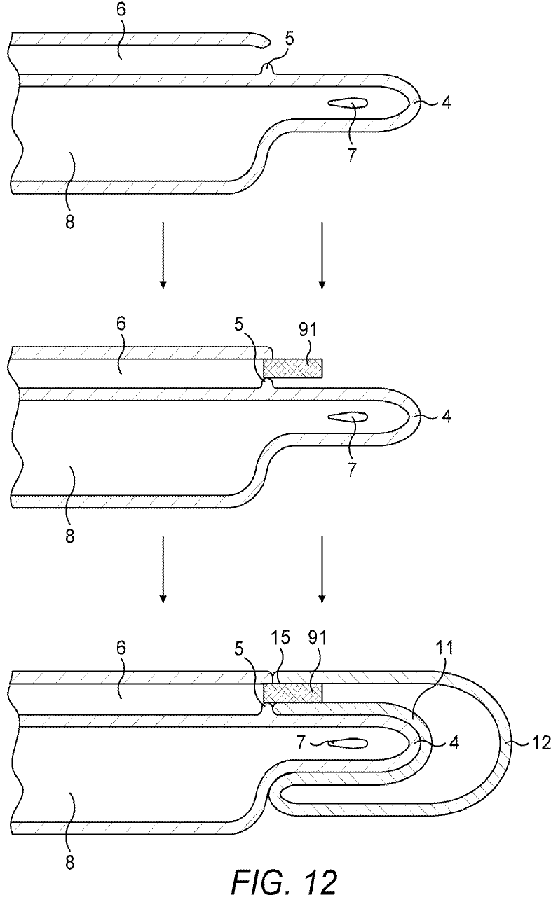

FIG. 12 shows a method of manufacturing the alternative embodiment illustrated in FIG. 11. The method of manufacture is outlined below.

A catheter shaft 1 is provided with a proximal region 2 and distal region 3. The distal region 3 of the shaft 1 ends in a tip 4. The shaft 1 has a drainage opening 7 located in the distal region 3 and on a side of the shaft 1. The drainage opening 7 is arranged so as to communicate with the drainage lumen 8 of the shaft 1. The shaft 1 also comprises an inflation opening 5. The inflation opening 5 is arranged so as to communicate with an inflation lumen 6.

A connection tube 91 is provided to the catheter shaft 1. As described above, in relation to FIG. 11, the connection tube 91 may take the form of a range of shapes depending on the design requirements for the catheter.

The connection tube 91 is inserted into the inflation opening 5 and attached to the inflation opening 5. The attachment may be provided by a friction fit, such as through thermal joining or elastic materials, or the attachment may be provided by adhesive bonding the connection tube 91 to the inflation opening 5. It is preferable to use adhesive as this may provide a more secure attachment. As described above, in relation to FIG. 11, the connection tube 91 is arranged to protrude into the inflation lumen 6 and to protrude out of the inflation opening 5.

An elastic walled conduit having an access opening to the interior is provided. In other words, a balloon, as described above in relation to FIG. 11, is provided to the catheter shaft 1.

The balloon is attached to the connection tube 91. In particular, the interior of the balloon is attached to the connection tube 91. The attachment may be provided by a friction fit, such as through thermal joining or elastic materials, or the attachment may be provided by adhesive bonding the connection tube 91 to the interior of the balloon. It is preferable to use adhesive as this may provide a more secure attachment. As described above in relation to FIG. 11, the connection tube 91 is arranged to protrude into the interior of the balloon.

In any of the embodiments, the outer surface of the catheter shaft 1 may define a recess in which the uninflated balloon can sit, such as the shoulders 92, 95 shown in FIG. 11. The recess may be sized so that the exterior of the uninflated balloon lies flush with the exposed surface of the catheter. This may help the catheter to be inserted through the urethra 16.

In any of the embodiments, additional layers of material may be provided over the balloon. For example, an additional web may be provided over the balloon in order to smooth the exterior surface of the catheter's distal end when the balloon is inflated. Alternatively, or in addition, there could be a further balloon located distally and/or laterally outward of the balloon described above.

As described above, the catheter is inserted into the patient through the urethra 16 into the bladder 17. This process may cause discomfort to the patient. To minimise the discomfort to the patient it is preferable for the catheter to be smooth. Smooth may be defined as a minimal level of surface roughness, or minimal features protruding from the from the catheter shaft 1. Additionally, a rough catheter may increase the likelihood of bacteria adhering to the catheter.

Protruding features are generally provided by the inherent design of the catheter and can be removed from the design. Surface roughness is often provided by the manufacturing process. Surface roughness is often produced during the piercing of the catheter to produce the openings 5, 7. The manufacturing method explained below aims to reduce the surface roughness of the catheter.

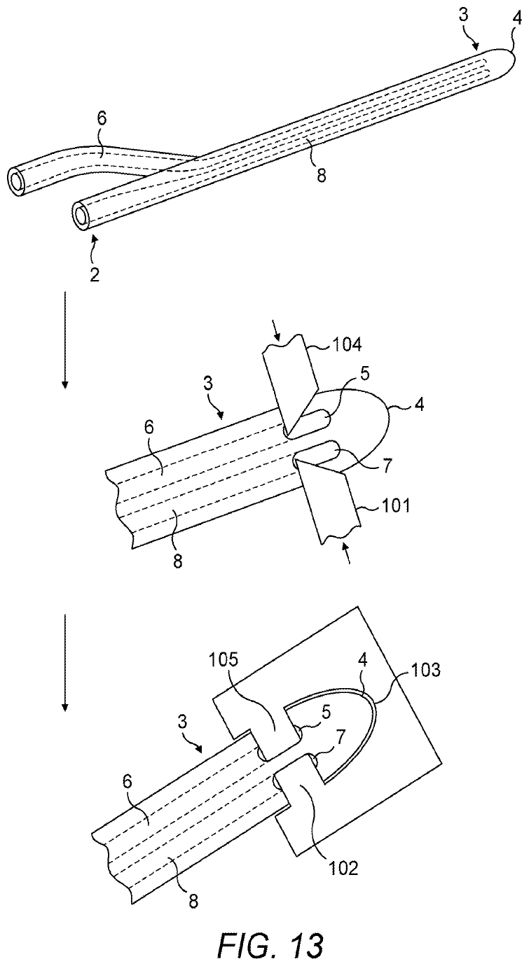

FIG. 13 shows an alternative manufacturing method that may be applied to any of the above-described catheter embodiments. The manufacturing method uses a first tool 101, a second tool 102, a third tool 103, a fourth tool 104 and a fifth tool 105. The first tool 101 and the fourth tool 104 may provide pressure. The second tool 102, third tool 103 and the fifth tool 105 may provide heat and pressure. The method of manufacturing is outlined below.

A catheter shaft 1 is provided with a proximal region 2 and distal region 3. The distal region 3 of the shaft 1 ends to a tip 4. The shaft 1 has a drainage lumen 8 extending along the shaft 1 and to the tip 4. The drainage lumen 8 is defined by walls of the shaft 1. The shaft 1 may also have an inflation lumen 6 extending along the shaft 1 to the tip 4.

A first tool 101 and a fourth tool 104 are introduced to a wall of the shaft 1 in the distal region 3. The first tool 101 and the fourth tool 104 may be provided at different times or the same time and be provided on the same tool holder or be provided by the same tool.

The first tool 101 and the fourth tool 104 are used to pierce a wall of the shaft 1 in the distal region 104. The first tool 101 pierces the wall of the shaft 1 in the location of the drainage lumen 8 to form the drainage opening 7. The fourth tool 104 pierces the wall of the shaft 1 in the location of the inflation lumen 6 to form the inflation opening 5.

The drainage opening 7 is preferably located on a side of the shaft 1 for the advantages explained in relation to any of the above catheter embodiments.

A second tool 102 is introduced into the drainage opening 7, a third tool 103 is introduced around the tip 4 of the shaft 1 and a fifth tool 105 is introduced to the inflation opening 5. The second tool 102, third tool 103 and the fifth tool 105 may be provided at the same time and on the same tool holder or be provided by the same tool. For example, the three tools maybe be provided in a ‘clam-shell’ type tool that both inserts into and encloses the distal region 3 of the shaft 1, as shown in FIG. 13.

The second tool 102 and fifth tool 105 may be arranged so as to increase the size of the drainage opening 7 and the inflation opening 5 respectively. This increase is size may be provided by the tool 102, 105 being larger than the respective opening 7, 5. Alternatively, the tool 102, 105 may be smaller than the opening and expand once it has been inserted into the opening 7, 5.

The distal region 3 of the shaft 1 is subjected to a temperature increase. The heating to provide the temperature increase may be provided by one or more of the second tool 102, third tool 103 and the fifth tool 105 or the tool holder. Alternatively, the tool may not provide the heat and the heat may be provided by a separate heater which may in turn heat one or more of the tools 102, 103, 105.

The temperature increase is sufficient to smooth the distal region 3 of the shaft 1. In other words, the temperature is raised to a level above the softening temperature of the shaft 1 material. At this level the combination of the pressure from the tools 102, 103, and 105 and the softened state of the shaft 1 material is sufficient for the shaft 1 material to flow and follow the surface of the tools 102, 103, and 105. Preferably, the tools 102, 103, and 105 are sufficiently smooth so that once the shaft 1 material has flowed the distal end 3 of the shaft 1 will adopt the same smoothness.

The distal region 3 of the shaft 1 includes the drainage opening 7, the inflation opening 5, and the tip 4. The drainage opening 7 and inflation opening 5 may comprise radiused or chamfered edges. The tools 102 and 105 may correspondingly comprise radiused or chamfered corners to form the radiused or chamfered edges of the openings 5, 7. The tip 4 may comprise a rounded convex shape, as described in relation to the embodiments above. The tool 103 may correspondingly comprise a rounded concave shape to form the rounded convex shape of the tip 4.

The distal region 3 of the shaft 1 is held at the increased temperature for a predetermined period of time. The period of time is sufficient for the material of the shaft 1 to follow into the mould provided by the tools 102, 103, 105. This period of time may vary depending on the material type and the thickness of the material of the shaft 1. In this embodiment, preferably the material of the shaft 1 is a polymer. More preferably the material of the shaft 1 is a thermoplastic polymer such that the shaft material 1 becomes softer with increased temperature.

The distal region 3 of the shaft 1 can then be subjected to a temperature decrease. The decreased temperature is lower than the softening temperature of the shaft 1 material. In this way, once the shaft 1 has cooled, the shaft 1 material will harden and take the form of the mould provided by the tools 102, 103, 105.

It may be appreciated that the catheter may comprise any of the features mentioned in relation to the above catheter embodiments.

The shaft of the catheter may be formed of a material such as polyurethane, a silicone elastomer or latex. A polyurethane catheter shaft can be more rigid than comparable rubber catheter shafts. This can allow the shaft to have a larger urine carrying capacity without sacrificing rigidity for insertion.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1-58. (canceled)

59. A catheter comprising:
a shaft having a proximal region and a distal region, the distal region terminating in a tip;
a balloon located at a distal end of the shaft;
an inflation opening located in the distal region of the shaft, the inflation opening communicating with an inflation lumen of the shaft;
a connection tube located in the distal region of the shaft, the connection tube communicating with the inflation opening and the interior of the balloon;
wherein the connection tube protrudes from the inflation opening and protrudes into the interior of the balloon.

60. The catheter as claimed in claim 59, wherein the connection tube protrudes at least 2 mm from the inflation opening.

61. The catheter as claimed in claim 59, wherein the interior of the balloon is secured to the connection tube.

62. The catheter as claimed in claim 59, wherein the connection tube protrudes into the interior of the balloon by at least 2 mm.

63. The catheter as claimed in claim 59, wherein at least a part of the connection tube is located inside the inflation lumen.

64. The catheter as claimed in claim 63, wherein the connection tube extends into the inflation lumen by at least 2 mm.

65. The catheter as claimed in claim 59, wherein the shaft comprises a first shoulder in the distal region of the shaft, the first shoulder being recessed relative to the external diameter of the shaft.

66. The catheter as claimed in claim 65, wherein the inflation opening is located on the first shoulder of the shaft.

67. The catheter as claimed in claim 59, wherein the balloon comprises a first region secured to the connection tube, a second region secured to the shaft and an elastic-walled and/or flexible-walled conduit extending between the first region and the second region.

68. The catheter as claimed in claim 67, wherein the shaft comprises a first shoulder in the distal region of the shaft, the first shoulder being recessed relative to the external diameter of the shaft, and wherein the shaft comprises a second shoulder on an opposing side of the shaft to the first shoulder, the second shoulder being recessed relative to the external diameter of the shaft, the second region of the balloon being secured to the second shoulder.

69. The catheter as claimed in claim 59, wherein the conduit extends over the tip of the shaft.

70. The catheter as claimed in claim 59, further comprising a drainage opening located at the distal end of the shaft, the drainage opening communicating with a drainage lumen of the shaft.

71. The catheter as claimed in claim 70, wherein the drainage opening of the shaft is located on a side of the shaft.

72. The catheter as claimed in claim 70, wherein the balloon comprises a first region secured to the connection tube, a second region secured to the shaft and an elastic-walled and/or flexible-walled conduit extending between the first region and the second region, and wherein at least part of the first region and at least part of the second region are located proximally of the drainage opening.

73. The catheter as claimed in claim 59, wherein the catheter is an indwelling urinary catheter configured to be retained in the bladder of a patient.

74. A method for manufacturing a catheter comprising:
providing a shaft having a proximal region and a distal region, the distal region terminating in a tip, a drainage opening located in the distal region of the shaft, the drainage opening communicating with a drainage lumen of the shaft and an inflation opening located in the distal region of the shaft, the inflation opening communicating with an inflation lumen of the shaft;
providing a connection tube;
securing the connection tube inside the inflation opening;
providing an elastic-walled conduit having an access opening to the interior thereof;
securing the access opening of the conduit around the connection tube.

75. The method as claimed in claim 74, wherein the connection tube protrudes at least 2 mm from the inflation opening.

76. The method as claimed in claim 74, wherein the connection tube protrudes into the interior of the conduit.

77. The method as claimed in claim 74, wherein the interior of the conduit is secured to the connection tube.

78. The method as claimed in claim 74, wherein at least a part of the connection tube is located inside the inflation lumen.
